(19) United States
(12) Patent Application Publication
(10) Pub. No.: US 2009/0209975 A1
(43) Pub. Date: Aug. 20, 2009

(54) Catheter Shape Forming System

(75) Inventors: Zoran Milijasevic, Bayview (AU); Norman Booth, Wattle Grove (AU)

Correspondence Address: MORRISON & FOERSTER LLP, 755 PAGE MILL RD, PALO ALTO, CA, 94304-1018, US

(73) Assignee: CathRx Ltd.

(21) Appl. No.: 11/922,656

(22) PCT Filed: Jun. 23, 2006
(86) PCT No.: PCT/AU2006/000891

§ 371 Date: Apr. 6, 2009

Related U.S. Application Data
(60) Provisional application No. 60/693,688, filed on Jun. 24, 2005.

(51) Int. Cl. A61B 18/18

(52) U.S. Cl. 606/129

(57) ABSTRACT

A catheter assembly (10) includes a handle body (12) having a proximal end (14) and a distal end. An introducer carrier (18) is displaceably arranged relative to the handle body (12). An electrode sheath component is arranged at a distal end of the handle body (12), the electrode sheath component comprising at least one pair of limbs interconnected by a bridging portion, at least the bridging portion carrying at least one electrode. An elongate shape forming component (40) is received in a lumen of the electrode sheath component, the shape forming component (40) having at least one predetermined shape formed along its length. An introducer component (22) is carried on the introducer carrier (18) of the handle body (12). The electrode sheath component is received within the introducer component (22) prior to use with at least the bridging portion of the electrode sheath component being extended out of a distal end of the introducer component (22) to be exposed for use. The arrangement is such that movement between at least two of the components relative to each other results in a change of shape being imparted to the exposed bridging portion of the electrode sheath component.

### CROSS-REFERENCE TO RELATED APPLICATIONS

The present application claims priority from United States of America Provisional Patent Application No. 60/693,688 filed on 24 Jun. 2005, the contents of which are incorporated herein by reference.

### FIELD OF THE INVENTION

This invention relates, generally, to the field of catheters and, more particularly, to a catheter assembly and to a catheter handle and a catheter stylet for use with the catheter assembly.

### BACKGROUND TO THE INVENTION

In the heat treatment of a biological site in a patient's body, surface irregularities at the site may impede electrode-tissue contact. This could result in the treatment regime not being as efficient as would otherwise be the case. In order to overcome or accommodate such surface irregularities, it would be useful to be able to apply pressure on the electrode, or electrodes, being used to carry out the heat treatment.

In addition, a biological site in a patient's body to be treated may be located in a position difficult to reach or the nature of the treatment necessary may require versatile catheter arrangements.

The Applicant has filed an International Patent Application for an electrical lead under International Patent Application Number PCT/AU01/01339 dated 19 Oct. 2001. The contents of that application are incorporated in this specification by reference. The electrical lead forming the subject matter of the International Patent Application is suitable as an electrode sheath of a catheter to be used in the heat treatment of the biological site in a patient's body. It lends itself to this application due to the fact that such an electrode sheath has an unimpeded lumen into which items to assist in manoeuvring and manipulating the electrode sheath can be inserted while retaining a narrow diameter sheath which is beneficial in steering the catheter through the vascular system of the patient to the site of interest.

### SUMMARY OF THE INVENTION

According to a first aspect of the invention, there is provided a catheter assembly which includes:

a handle body having a proximal end and a distal end;

an introducer carrier displaceably arranged relative to the handle body;

an electrode sheath component arranged at a distal end of the handle body, the electrode sheath component comprising at least one pair of limbs interconnected by a bridging portion, at least the bridging portion carrying at least one electrode;

an elongate shape forming component received in a lumen of the electrode sheath component, the shape forming component having at least one predetermined shape formed along its length; and

an introducer component carried on the introducer carrier of the handle body, the electrode sheath component being received within the introducer component prior to use with at least the bridging portion of the electrode sheath component being extended out of a distal end of the introducer component to be exposed for use, the arrangement being such that movement between at least two of the components relative to each other results in a change of shape being imparted to the exposed bridging portion of the electrode sheath component.

It will be appreciated that, generally, the shaped part of the shape forming component would be arranged within the lumen of the electrode sheath component to be in register with the bridging portion to impart that shape to the bridging portion when the bridging portion is exposed by ejection of the bridging portion from the distal end of the introducer component.

For the sake of brevity, the electrode sheath component will be referred to below as the “electrode sheath” and the introducer component will be referred to below as the “introducer”.

The handle body may carry a steering control mechanism. The steering control mechanism, preferably, comprises a plurality of actuators to facilitate omnidirectional steering of a distal end of the introducer.

As indicated above, the electrode sheath may comprise a substantially U-shaped arrangement with a pair of limbs interconnected by the bridging portion. The limbs may extend parallel to each other and free ends of the limbs, forming a proximal end of the electrode sheath, may be received in a receiving formation at a distal end of the handle body. The receiving formation may be an axially extending bore in which the proximal end of the electrode sheath is received. To improve the modularity of the assembly, the proximal end of the electrode sheath may be removably received in the bore of the handle body.

A proximal end of the bore may open into an axially extending passage defined in the handle body. The passage may carry electrical leads from at least one electrical connector arranged at the proximal end of the handle body to connect to electrodes of the electrode sheath via conductors in the electrode sheath.

The electrode sheath may comprise two separate elements which are connected together at the bridging portion or, instead, the electrode sheath could be a single element bent into a substantially hairpin shape at least when the bridging portion of the electrode sheath is withdrawn into the introducer.

The handle body may include a displacement mechanism for displacing the shape forming component relative to the other components. In one form, the displacement mechanism may include at least one introduction port through which the shape forming component is inserted into the lumen of the electrode sheath. Preferably, at least two such ports are provided with free ends of the shape forming component extending out of the handle body through the ports. The displacement mechanism may further comprise a gripping formation may be arranged on each free end of the shape forming component to facilitate manipulation of the shape forming component relative to the electrode sheath.

Instead, the displacement mechanism may include a displaceable element carried on the handle body, the shape forming component being connected to the displaceable element such that manipulation of the displaceable element causes displacement of the shape forming component relative to the other components. The displaceable element may, for example, be in the form of a thumb wheel, or the like, which may, if necessary, be suitably geared to achieve the required displacement.

The shape forming component may be in the form of a stylet comprising an elongate element, at least a part of which is of a shape memory material. The at least one shape of the shape memory component may therefore be formed in that part of the elongate element of the shape memory material. The shape memory material may, for example, be a superlastic alloy. For lubrication purposes, the shape forming component may be coated with a friction reducing coating such as a polytetrafluoroethylene coating. In addition, or instead, the lumen of the electrode sheath may contain a friction reducing material such as a silicone oil or equivalent material.

A plurality of predetermined, different shapes may be formed at longitudinally spaced intervals along the part of the elongate element of the stylet. The stylet may be displaceable relative to the electrode sheath to move the desired shape into register with the bridging portion of the electrode sheath.

To facilitate displacement of the stylet relative to the electrode sheath, a hinge-like arrangement may be formed between adjacent shapes of the stylet. The hinge-like arrangement may be implemented in numerous ways. For example, the hinge-like arrangement may be a region of reduced thickness of the elongate element. The region of reduced thickness may be formed by scalloping the elongate element at the desired location or locations. The scalloped region/s of the elongate element may, if necessary, be insulated by being covered with a suitable sleeve which may be of a heat shrink material. Instead, the elongate element may be formed in sections with the sections being joined in end-to-end relationship by lengths of polymer material such as, for example, heat shrink plastics tubing. Still further, the hinge-like arrangement may be formed by a work treated region of the elongate element. The work treated region may be an annealed region.

In another embodiment, the elongate element of the stylet may be of two parts, one part being of superlastic alloy with the other part being of a polymeric material and the parts being arranged in end-to-end relationship. In use, while the bridging portion of the electrode sheath is still within the introducer, the length of polymeric material may extend through the bridging portion. Prior to the bridging portion being ejected from the introducer, the polymeric part of the stylet may be pulled to draw the required shape into position relative to the bridging portion of the electrode sheath component.

The introducer carrier may be in the form of a nosecone-like structure which is axially slidable relative to the distal end of the handle body.

According to a second aspect of the invention, there is provided a catheter handle which includes:

a handle body having a proximal end and a distal end;

an introducer carrier displaceably arranged relative to the distal end of the handle body; and

a displacement mechanism associated with the handle body by means of which a shape forming component can be manipulated relative to the handle body.

The displacement mechanism may include at least one introduction port through which the shape forming component is able to be inserted into a lumen of an electrode sheath attached, in use, to a distal end of the handle body. Preferably, at least two such ports are provided with free ends of the shape forming component extending out of the handle body through the ports. The displacement mechanism may further include a gripping formation arranged on each free end of the shape forming component to facilitate manipulation of the shape forming component relative to the electrode sheath.

Instead, the displacement mechanism may include a displaceable element carried on the handle body, the shape forming component, in use, being connected to the displaceable element such that manipulation of the displaceable element causes displacement of the shape forming component relative to the electrode sheath. The displaceable element may, for example, be in the form of a thumb wheel or the like, which may, if necessary, be suitably geared to achieve the required displacement.

According to a third aspect of the invention, there is provided a catheter stylet which includes:

an elongate element, at least a part of which is of a shape memory material; and

a plurality of predetermined shapes formed at spaced intervals along the length of the part of the elongate element.

To facilitate displacement of the stylet relative to an electrode sheath, a hinge-like arrangement may be formed between adjacent shapes of the stylet. The hinge-like arrangement may be implemented in numerous ways. For example, the hinge-like arrangement may be a region of reduced thickness of the elongate element. Instead, the elongate element may be formed in sections with the sections being joined in end-to-end relationship by lengths of polymer material such as, for example, heat shrink plastics tubing. Still further, the hinge-like arrangement may be formed by a work treated region of the elongate element. The work treated region may be an annealed region.

In another embodiment, the stylet may be of two parts, one part being of superlastic alloy with the other part being of a polymeric material and the parts being arranged in end-to-end relationship. In use, while a bridging portion of the electrode sheath is still within an introducer, the length of polymeric material may extend through the bridging portion. Prior to the bridging portion being ejected from the introducer, the polymeric part of the stylet may be pulled to draw the required shape into position relative to the bridging portion of the electrode sheath component.

### BRIEF DESCRIPTION OF THE DRAWINGS

FIG. 1 shows a schematic, plan view of a catheter assembly, in accordance with an embodiment of the invention, with an electrode sheath withdrawn into an introducer of the assembly;

FIG. 2 shows a three dimensional view of a part of a distal part of a catheter handle, also in accordance with an embodiment of the invention;

FIG. 3a shows a schematic, sectional side view of a part of a distal end of a catheter of the assembly prior to ejection from an introducer of the assembly;

FIG. 3b shows a schematic, sectional side view of the part of the distal end of the catheter of the assembly after ejection from the introducer of the assembly;

FIGS. 4a-4c show schematic side views of different shapes imparted to the distal end of the catheter by a catheter stylet received in an electrode sheath of the catheter;

FIGS. 5a and 5b show schematically one example of how the shape at the distal end of the catheter can be manipulated;

FIGS. 6a and 6b show schematically another example of how the shape at the distal end of the catheter can be manipulated;

FIG. 7 shows a schematic side view of a different displacement mechanism of the catheter handle for displacing the stylet relative to the electrode sheath of the catheter;

FIG. 8 shows a schematic side view of a first example of a catheter stylet, in accordance with yet a further embodiment of the invention;

FIG. 9 shows a schematic side view of a second example of a catheter stylet, in accordance with the yet further embodiment of the invention; and

FIG. 10 shows a schematic side view of a third example of a catheter stylet, in accordance with the yet further embodiment of the invention.

### DESCRIPTION OF EXEMPLARY EMBODIMENTS

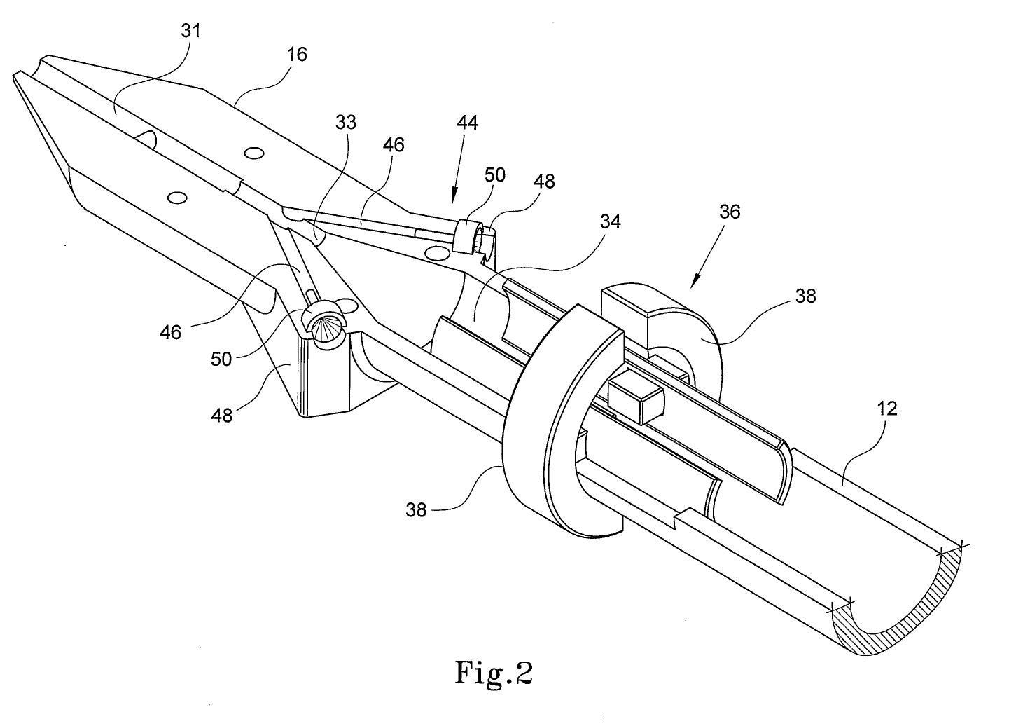

In the drawings, reference numeral 10 generally designates a catheter assembly, in accordance with an embodiment of the invention. The assembly 10 includes an elongate handle body 12 having a proximal end 14 and a distal end 16 (FIG. 2). An introducer carrier 18 is arranged on the distal end 16 of the body 12 and is displaceable relative to the body 12 in the direction of arrows 20.

An introducer component, or introducer, 22 in the form of an elongate sleeve, is carried by the introducer carrier 18. The introducer 22 is mounted on a distal end of the introducer carrier 18.

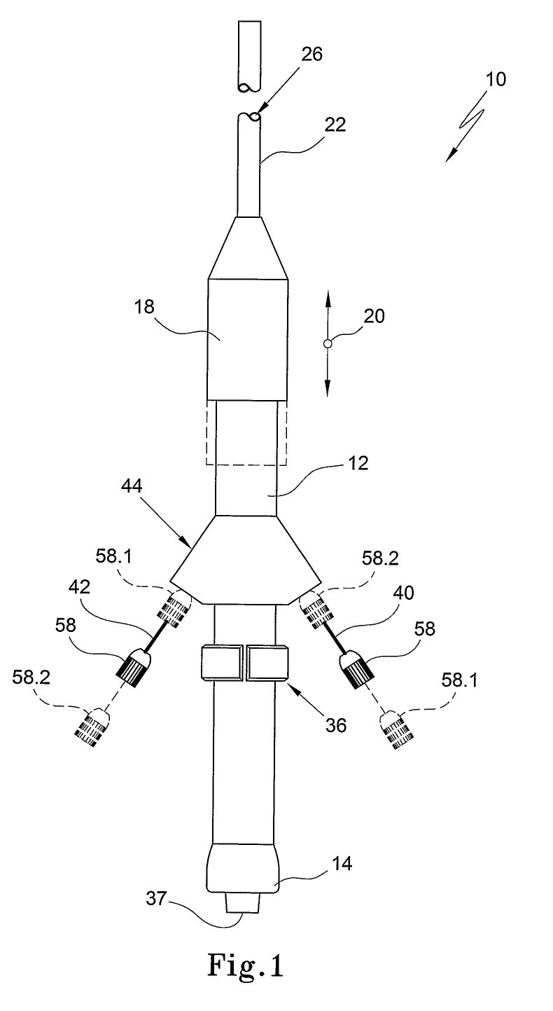

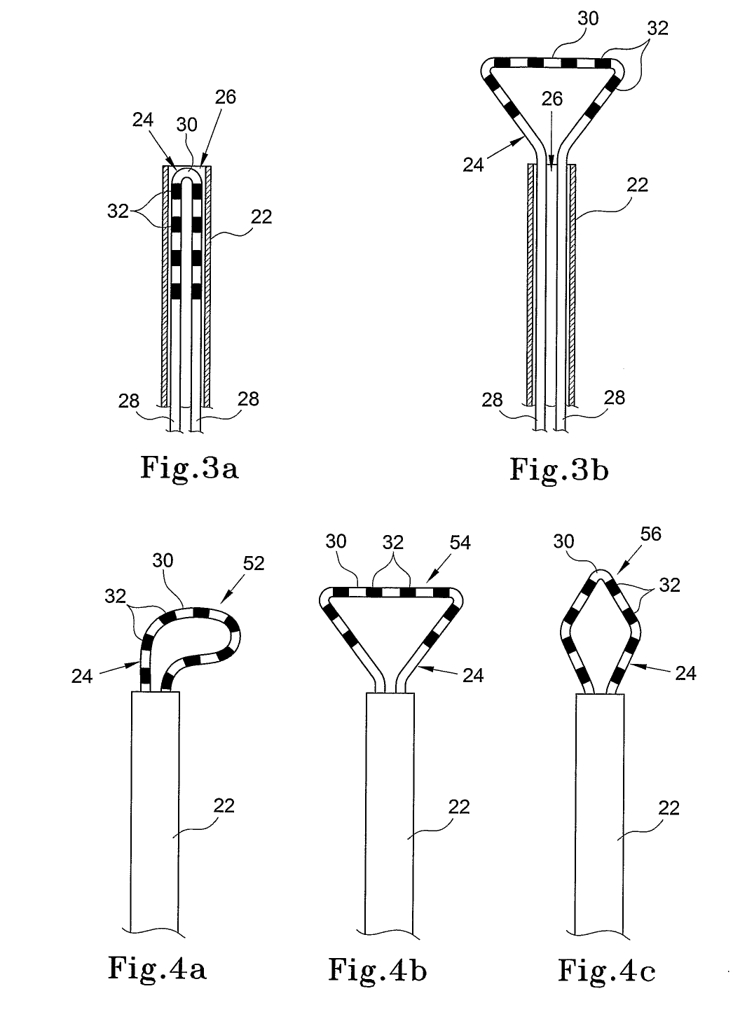

An electrode sheath component, or electrode sheath, 24 (not shown in FIG. 1 of the drawings but shown, for example, in FIGS. 3a and 3b of the drawings) is mounted to the distal end 16 of the handle body 12 and is received within a passage 26 of the introducer 22.

Prior to introduction of the introducer 22 into a patient's body, the electrode sheath 24 is withdrawn into the passage 26 of the introducer 22 as shown in FIG. 3a of the drawings. It is to be noted that the electrode sheath 24 adopts a hairpin-shape and has a pair of spaced limbs 28 interconnected by a bridging portion 30. At least the bridging portion 30 of the electrode sheath 24 carries a plurality of electrodes 32. Each electrode 32 is connected to an electrical conductor (not shown) embedded in a wall of the electrode sheath 24 in accordance with the method described in the Applicant's International Patent Application No. PCT/AU01/01339, referenced above.

The limbs 28 of the electrode sheath 24 are received in an axially extending bore 30 defined in the distal end 16 of the handle body 12. The handle body 12 is made up of a pair of mating shells, one of which is shown at 32 in FIG. 2 of the drawings.

The electrical conductors of the electrode sheath 24 connect to conductors (not shown) extending in an axial passage 34 which is in communication with a proximal end of the bore 30. The conductors in the passage 34 connect to a connector 36 (FIG. 1) arranged at the proximal end 14 of the handle body 12.

The handle body 12 carries a steering control mechanism 36. In the embodiment shown in FIG. 2 of the drawings, the steering control mechanism 36 comprises a pair of actuators, or slides, 38 which are axially displaceable along the handle body 12 and to which steering control wires (not shown) for steering a distal end of the introducer 22 or the electrode sheath 24 are connected. With two such slides 38, bending of the distal end of the introducer 22 or the electrode sheath 24, as the case may be, occurs in-plane. However, the steering control mechanism 36 could include three slides 38 in which case omni-directional steering can be achieved.

The assembly 10 includes an elongate shape forming component, or stylet, 40. The stylet 40 comprises an elongate element 42 which is at least partly formed from a superlastic alloy such as nitinol. In the embodiment of the invention illustrated in FIGS. 1 and 2 of the drawings, the handle body 12 includes an introduction zone 44 via which the elongate element 42 of the stylet 40 is inserted into a lumen (not shown) of the electrode sheath 24. The introduction zone 44 comprises a pair of angled introducing ports 46 (FIG. 2). Each port 46 is defined in a backswept wing member 48 of the handle body 12. A funnel-shaped guide formation 50 is arranged at an entrance to each port 46 for guiding the elongate element 42 into the lumen of the electrode sheath 24.

The arrangement of the stylet 40 used in the embodiment shown in FIG. 1 of the drawings is, for example, as shown in FIG. 8 of the drawings. The elongate element 42 of the shape stylet 40 comprises three serially arranged shapes 52, 54 and 56. A grip 58 is arranged at each free end of the elongate element 42 of the stylet 40 with the grips 58 extending proximally from the introduction zone 44 of the handle body 12. When the middle shape, the shape 54, is in register with the bridging portion 30 of the elongate sheath 24, the grips 58 are equidistantly spaced from the introduction zone 44 of the handle body 12 as shown in solid lines in FIG. 1 of the drawings. To bring the shape 52 into register with the bridging portion 30 of the electrode sheath 24, the grips 58 are manipulated so that the grips 58 adopt the position shown at 58.1 in FIG. 1 of the drawings. Conversely, to bring the shape 56 into register with the bridging portion 30 of the electrode sheath 24, the grips are manipulated so that they adopt the position shown at 58.2 in FIG. 1 of the drawings.

At least one of the grips 58 may be removably secured to its associated end of the elongate element 42 to facilitate threading of the elongate element 42 through the lumen of the electrode sheath 24.

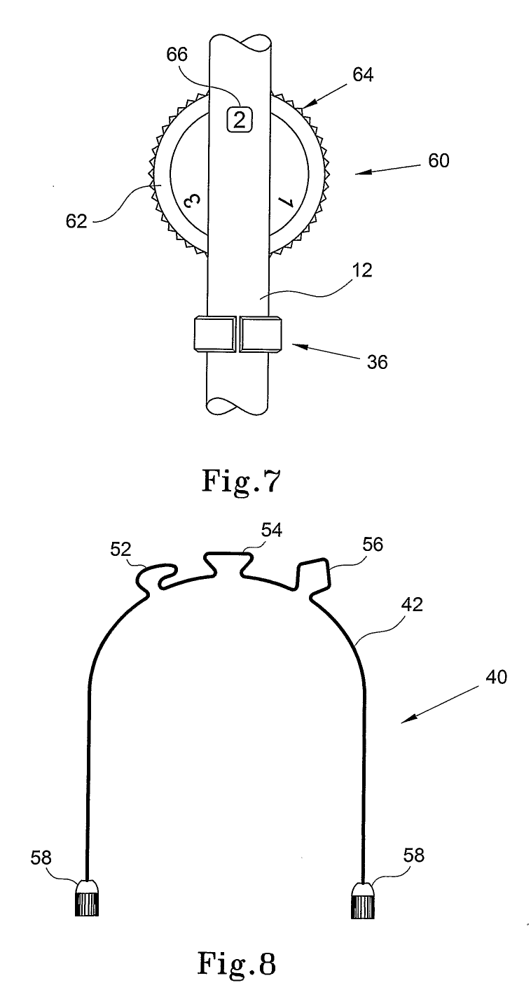

In FIG. 7 of the drawings, a further embodiment of a displacement mechanism 60 for displacing the stylet 40 relative to the electrode sheath 24 is shown. In this embodiment, the displacement mechanism 60 of the handle body 12 has a thumb wheel 62 with a knurled periphery 64. Ends of the stylet 40 are connected to the thumb wheel 62 at diametrically opposed locations. By rotating the thumb wheel 62, the appropriate shape 52, 54 or 56, as the case may be, is brought into register with the bridging portion 30 of the electrode sheath 24. The selected shape 52, 54 or 56 is shown in a window 66 defined in the handle body 12.

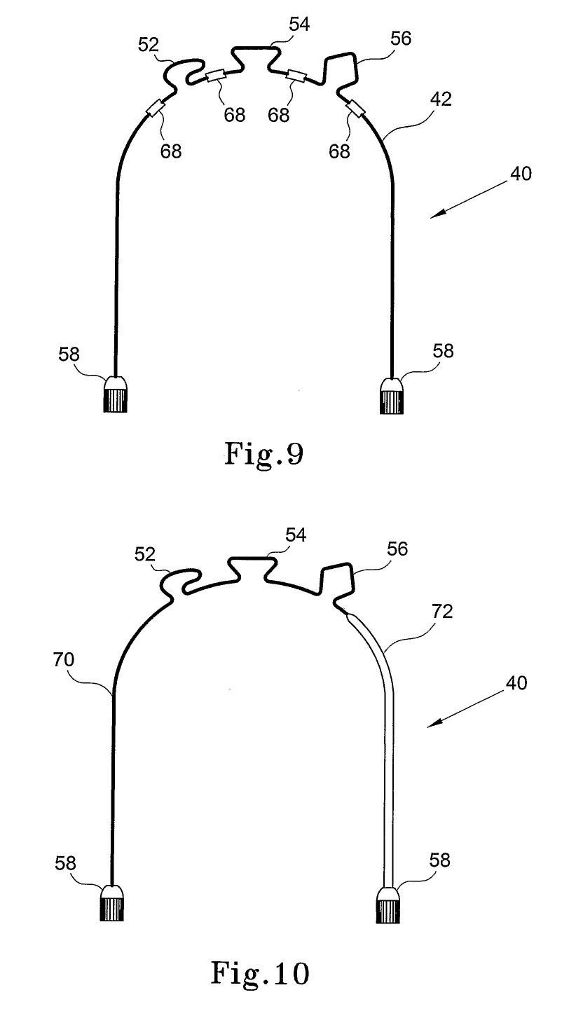

In FIGS. 9 and 10 of the drawings, different versions of the stylet 40 are shown. To facilitate ejection of the distal end of the electrode sheath 24 relative to the introducer 22, the stylet 40 may have “softer” sections formed in one of a number of ways. In the embodiment shown in FIG. 9 of the drawings, each shape 52, 54, 56 is separated from an adjacent portion of the elongate element 42 via a hinge-like arrangement 68. The hinge-like arrangement 68 comprises a length of polymer which is of greater flexibility than the superlastic alloy of the elongate element 42 of the stylet 40. The hinge-like arrangements 68 facilitate the manipulation of the stylet 40 relative to the electrode sheath 24.

Other forms of hinge-like arrangements could include regions of reduced thickness between adjacent shapes 52, 54, 56 or work treating, such as annealing, the elongate element 42 at least between the shapes 52, 54, 56.

In the embodiment shown in FIG. 9 of the drawings, a first part 70 of the stylet 40, including the shapes 52, 54, 56 is made from the superlastic alloy. A further part 72 is made of a polymer with the parts 70 and 72 being arranged in end-to-end relationship. Prior to ejection of the bridging portion 30 of the electrode sheath 24 from the distal end of the introducer 22, a distal end of the part 72 lies in register with the bridging portion 30. The required shape 52, 54, 56 is brought into register with the bridging portion 30 of the electrode sheath 24 prior to ejection of the bridging portion 30 from the introducer 22 by pulling on the part 72 in a proximal direction.

FIGS. 4a-4c show the shapes 52, 54, 56, respectively, once the bridging portion 30 has been ejected from the distal end of the introducer 22. To effect ejection of the bridging portion 30 of the electrode sheath 24 from the distal end of the introducer 22, the introducer carrier 18 is urged towards the proximal end 14 of the handle body 12 until it adopts the position shown in dotted lines in FIG. 1 of the drawings so that the selected shape 52, 54, 56 is ejected from the distal end of the introducer 22 for use.

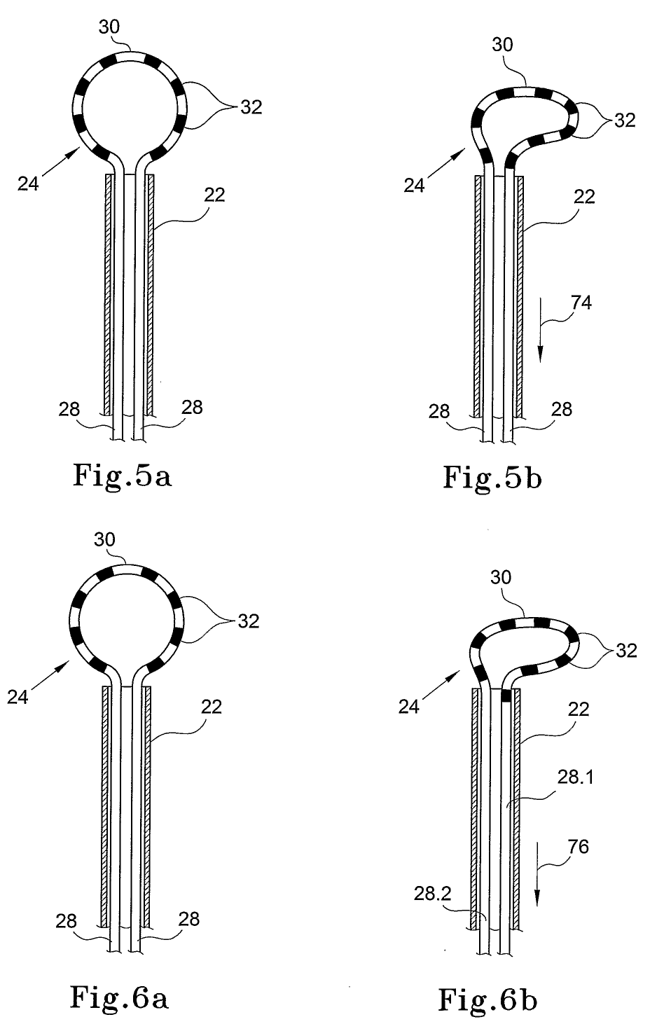

The embodiments described above relate to a stylet 40 having a plurality of shapes arranged at intervals along the length of the elongate element 42. In another embodiment of the invention, illustrated in FIGS. 5 and 6 of the drawings, the stylet 14 has a single shape. In the embodiment shown in FIG. 5 of the drawings, when the shape is in register with the bridging portion 30 of the electrode sheath 24, the shape imparted to the bridging portion 30 corresponds to the shape carried by the stylet 40 in an undistorted form. However, by pulling on the stylet in the direction of arrow 74, while retaining the position of the electrode sheath 24 relative to the introducer 22, the shape is distorted as shown in FIG. 5b of the drawings.

A similar effect can be achieved, as shown in FIG. 6 of the drawings, by displacing a limb 28.1 of the electrode sheath 24 relative to the introducer 22 while retaining a limb 28.2 in its original position. As the limb 28.1 is drawn into the introducer 22 in the direction of arrow 76, the original shape imparted to the electrode sheath, as shown in FIG. 6a of the drawings, is distorted until the bridging portion 30 of the electrode sheath 24 adopts the shape as shown in FIG. 6b of the drawings. In this embodiment, the stylet 40 is not moved relative to the electrode sheath 23. Rather, the electrode sheath 24 and the stylet 40 are moved in unison relative to the introducer 22.

In the case of the embodiment shown in FIGS. 1 to 4 and 8 to 10 of the drawings, by moving the stylet 40 relative to the electrode sheath 24, the appropriate shape can be brought into register with the bridging portion 30 of the electrode sheath. Thus, while the bridging portion 30 of the electrode sheath is withdrawn into the introducer 22, as shown, for example, in FIG. 3a of the drawings, the appropriate shape of the stylet 40 is brought into register with the bridging portion 30 of the electrode sheath 24.

To deliver a distal part of the catheter assembly 10 to a biological site, such as an atrium of a patient's heart, the introducer 22 is inserted into a femoral vein of the patient's body and is steered through the vascular system of the patient until the distal part of the introducer 22 is in proximity to the site to be treated.

The distal part of the introducer 22 is then withdrawn from the bridging portion 30 of the electrode sheath 24 by urging the introducer carrier 18 towards the proximal end 14 of the handle body 12. Withdrawal of the introducer 22 effectively ejects the bridging portion 30 of the electrode sheath 24 from the distal end of the introducer 22 and the selected shape 52, 54, 56 of the stylet 40 is imparted to the bridging portion 30 of the electrode sheath 24.

The exposed bridging portion 30 of the electrode sheath 24 can then be urged into contact with tissue at the site to be treated. Heat treatment of the tissue is effected by energising the electrodes 32. For example, in the treatment of heart arrhythmias, the electrodes 32 ablate the tissue using RF energy to form lesions in an effort to eradicate the arrhythmia.

Advantageously, the U-shape of the electrode sheath 24 allows greater pressure to be brought to bear on the bridging portion 30 than could be brought to bear on a loose, free end of an ablation catheter. This results in more intimate electrode-tissue contact by overcoming surface irregularities and, the Applicant believes, results in improved lesions being formed.

In the case of this embodiment of the invention, should the clinician desire to change the shape of the bridging portion 30 of the electrode sheath, for example, to gain access to a different part of the atrium or to form a different pattern of lesions, the clinician withdraws the bridging portion 30 into the introducer 22 by urging the introducer carrier 18 to the position shown in solid lines in FIG. 1 of the drawings. By appropriate manipulation of the grips 58 of the stylet 40, a different shape can be brought into register with the bridging portion 30 of the electrode sheath 24 whereafter the bridging portion 30 is again ejected from the distal end of the introducer 22 by displacing the carrier 18 towards the proximal end 14 of the handle body 12.

In the case of the embodiments shown in FIGS. 5 and 6 of the drawings, the clinician can change the shape of the bridging portion 30, either by moving the stylet 40 relative to the electrode sheath 24 or by moving the combination of the electrode sheath 24 and the stylet 40 relative to the introducer 22, as described above.

It is therefore an advantage of the invention that a catheter assembly 10 is provided which facilitates improved electrode-tissue contact, provides a clinician with greater flexibility in forming lesions or otherwise treating a site of a patient's body and which allows the clinician greater scope to form different lesion patterns.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A catheter assembly which includes:
a handle body having a proximal end and a distal end;
an introducer carrier displaceably arranged relative to the handle body;
an electrode sheath component arranged at a distal end of the handle body, the electrode sheath component comprising at least one pair of limbs interconnected by a bridging portion, at least the bridging portion carrying at least one electrode;
an elongate shape forming component received in a lumen of the electrode sheath component, the shape forming component having at least one predetermined shape formed along its length; and
an introducer component carried on the introducer carrier of the handle body, the electrode sheath component being received within the introducer component prior to use with at least the bridging portion of the electrode sheath component being extended out of a distal end of the introducer component to be exposed for use, the arrangement being such that movement between at least two of the components relative to each other results in a change of shape being imparted to the exposed bridging portion of the electrode sheath component.

2. The assembly of claim 1 in which the handle body carries a steering control mechanism.

3. The assembly of claim 1 in which the electrode sheath component comprises a substantially U-shaped arrangement with a pair of limbs interconnected by the bridging portion.

4. The assembly of claim 3 in which the limbs extend parallel to each other and free ends of the limbs, forming a proximal end of the electrode sheath, are received in a receiving formation at a distal end of the handle body.

5. The assembly of claim 4 in which the proximal end of the electrode sheath component is removably received in the receiving formation of the handle body.

6. The assembly of claim 1 in which the handle body includes a displacement mechanism for displacing the shape forming component relative to the other components.

7. The assembly of claim 6 in which the displacement mechanism includes at least one introduction port through which the shape forming component is inserted into the lumen of the electrode sheath component.

8. The assembly of claim 7 in which at least two such ports are provided with free ends of the shape forming component extending out of the handle body through the ports and in which the displacement mechanism further comprises a gripping formation arranged on each free end of the shape forming component.

9. The assembly of claim 6 in which the displacement mechanism includes a displaceable element carried on the handle body, the shape forming component being connected to the displaceable element such that manipulation of the displaceable element causes displacement of the shape forming component relative to the other components.

10. The assembly of claim 1 in which the shape forming component is in the form of a stylet comprising an elongate element, at least a part of which is of a shape memory material.

11. The assembly of claim 10 in which a plurality of predetermined, different shapes are formed at longitudinally spaced intervals along the part of the elongate element of the stylet.

12. The assembly of claim 11 in which, to facilitate displacement of the stylet relative to the electrode sheath component, a hinge arrangement is formed at least between adjacent shapes of the stylet.

13. The assembly of claim 11 in which the elongate element of the stylet is of two parts, one part being of superlastic alloy with the other part being of a polymeric material and the parts being arranged in end-to-end relationship.

14. The assembly of claim 1 in which the introducer carrier is in the form of a nosecone structure which is axially slidable relative to the distal end of the handle body.

15. A catheter handle which includes:
a handle body having a proximal end and a distal end;
an introducer carrier displaceably arranged relative to the distal end of the handle body; and
a displacement mechanism associated with the handle body by means of which a shape forming component can be manipulated relative to the handle body.

16. The handle of claim 15 in which the displacement mechanism includes at least one introduction port through which the shape forming component is able to be inserted into a lumen of an electrode sheath attached, in use, to a distal end of the handle body.

17. The handle of claim 16 in which at least two such ports are provided with free ends of the shape forming component extending out of the handle body through the ports, the displacement mechanism including a gripping formation arranged on each free end of the shape forming component to facilitate manipulation of the shape forming component relative to the electrode sheath.

18. The handle of claim 15 in which the displacement mechanism includes a displaceable element carried on the handle body, the shape forming component, in use, being connected to the displaceable element such that manipulation of the displaceable element causes displacement of the shape forming component relative to the electrode sheath.

19. A catheter stylet which includes:
an elongate element, at least a part of which is of a shape memory material; and
a plurality of predetermined shapes formed at spaced intervals along the length of the part of the elongate element.

20. The stylet of claim 19 in which a hinge arrangement is formed at least between adjacent shapes of the stylet.

21. The stylet of claim 19 which is of two parts, one part being of superlastic alloy with the other part being of a polymeric material and the parts being arranged in end-to-end relationship.
